(19) United States
(12) Patent Application Publication
(10) Pub. No.: US 2012/0010205 A1
(43) Pub. Date: Jan. 12, 2012

(54) NOVEL QUINOLINE ESTERS USEFUL FOR TREATING SKIN DISORDERS

(75) Inventors: Ronald Charles Bernotas, Wayne, PA (US); Robert Singhaus, Pottstown, PA (US); Sunil Nagpal, Collegeville, PA (US); Catherine Thompson, Perkiomenville, PA (US)

(73) Assignee: Wyeth LLC, Madison, NJ (US)

(21) Appl. No.: 13/177,908

(22) Filed: Jul. 7, 2011

Related U.S. Application Data
(60) Provisional application No. 61/362,320, filed on Jul. 8, 2010.

(51) Int. Cl. A61K 31/47; C07D 413/12; C07D 215/14; A61Q 19/08; A61P 17/00; A61P 17/06; A61P 17/02; A61K 31/5377; A61K 8/49

(52) U.S. Cl. 514/235.2; 514/311; 544/128; 546/172

(57) ABSTRACT

Disclosed are quinoline esters of Formula (I):

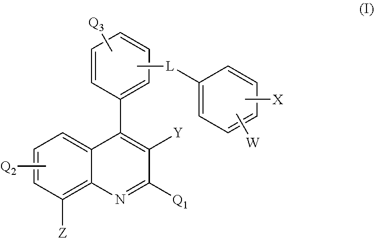

which are useful as Liver X receptors (LXR) modulators. Pharmaceutical compositions containing quinoline esters of Formula (I) and the use of quinoline esters of Formula (I) in the safe treatment of various skin disorders are also disclosed. Methods for preparing and using quinoline esters are further described.

### FIELD OF THE INVENTION

The present invention relates to quinoline esters that are effective as Liver X receptors (LXR) modulators. The present invention also relates to compositions comprising LXR modulators, and to methods for preparing such compounds. The invention further relates to the use of quinoline esters in the safe treatment of various skin disorders and conditions.

### BACKGROUND OF THE INVENTION

Skin is subject to deterioration through dermatological disorders, environmental abuse (wind, air conditioning, central heating) or through the normal ageing process (chronoageing) which may be accelerated by exposure of skin to sun (photoageing). In recent years the demand for safer and non-toxic drugs for treating skin disorders has grown enormously.

Liver X receptors (LXRs), originally identified from liver as orphan receptors, are members of the nuclear hormone receptor super family and are expressed in skin, for example in keratinocytes, and granulocytes. LXRs are ligand-activated transcription factors and bind to DNA as obligate heterodimers with retinoid X receptors (RXRs). LXRs activated by oxysterols (endogenous ligands) display potent anti-inflammatory properties in vitro and in vivo. Topical application of LXR ligands inhibits inflammation in murine models of contact (oxazolone-induced) and irritant (TPA-induced) dermatitis. Recently, LXRa receptor activators have been reported, e.g., WO 98/32444, have a therapeutic application in the restoration of the skin's barrier function, the induction of differentiation and the inhibition of proliferation.

Numerous compounds having LXR modulator activity been proposed and explored as potential pharmaceuticals because of such activity. In practice, however, they have not been clinically acceptable because of various side effects. According to our invention, a novel subclass of quinoline esters having LXR modulating activity is useful in treating various dermatological disorders and conditions without resulting in unacceptable side effects. Our approach draws upon the known concept of “soft drugs” (N. S. Bodor, U.S. Pat. No. 6,610,675). “Soft drugs” are biologically active chemical compounds (drugs) which might structurally resemble known active drugs (soft analogues) or could be entirely new types of structures, but which are characterized by in vivo destruction (metabolism) to nontoxic moieties, after they achieve their therapeutic role.

### SUMMARY OF THE INVENTION

The present invention provides compounds of Formula (I):

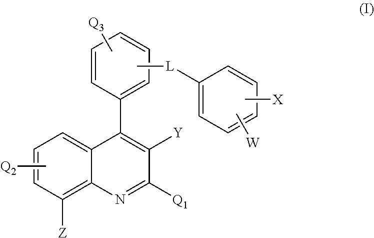

or a pharmaceutically acceptable salt thereof; wherein

Z is halogen or alkyl; wherein each alkyl is optionally substituted with halogen;

Y is H, alkyl, aryl, heteroaryl, cycloalkyl, heterocycloalkyl, CN; wherein each alkyl or aryl is optionally substituted with alkyl, or aryl;

Q₁, Q₂, O₃ are each independently H, halogen, alkyl, or aryl; wherein each alkyl, or aryl is optionally substituted with alkyl, or aryl;

L is OC(O), C(O)O, CH₂C(O)O, OC(O)CH₂;

W is H, halogen or alkyl;

X is H, alkyl, S(O)ₙR₁, SO₂NR₂R₃, CONR₄R₅, C(R₆)₂OR₇, CN; wherein each alkyl, S(O)ₙR₁, SO₂NR₂R₃, CONR₄R₅, or C(R₆)₂OR₇ is optionally substituted with alkyl, SO₂alkyl or SO₂aryl, or SO₂heteroaryl; wherein

R₁ is alkyl, aryl, heteroaryl or cycloalkyl;

R₂ and R₃ are each independently H, alkyl or heteroaryl;

R₄ and R₅ are each independently H or alkyl;

R₆ and R₇ are each independently H or alkyl; and

n is 1 or 2.

The present invention also provides a pharmaceutical composition comprising an effective amount of one or more compounds of Formula (I) or a pharmaceutically acceptable salt thereof and a pharmaceutically acceptable carrier.

The present invention also provides a method for treating a skin disorder in a patient comprising administering to a patient in need thereof an effective amount of a compound of Formula (I) or a pharmaceutically acceptable salt thereof or a pharmaceutical composition comprising an effective amount of a compound of Formula

In another embodiment, the skin disorder is selected from the group consisting of psoriasis, atopic dermatitis, skin wounds, skin aging, photoaging and wrinkling.

In other embodiment, the treatment of a skin disorder further comprises administering an additional therapeutic agent.

### DETAILED DESCRIPTION OF THE INVENTION

The present invention is related to quinoline esters of Formula (I), which are effective as Liver X receptors (LXR) modulators. The present invention is also related to compositions comprising LXR modulators, and to methods for preparing such compounds. The quinoline esters of the invention and their polymorphs, solvates, esters, tautomers, diastereomers, enantiomers, pharmaceutically acceptable salts or prodrugs show utility in the safe treatment of various skin disorders and conditions.

### DEFINITIONS

Before describing the present invention in detail, it is to be understood that this invention is not limited to specific compositions or process steps, as such may vary. It should be noted that, as used in this specification and the appended claims, the singular form “a”, “an” and “the” include plural references unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which this invention is related. The following terms are defined for purposes of the invention as described herein.

As used herein, unless otherwise noted, “alkyl” whether used alone or as part of a substituent group refers to a saturated straight and branched carbon chain having 1 to 20 carbon atoms or any number within this range, for example, 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Where so indicated, alkyl groups can be optionally substituted. In substituent groups with multiple alkyl groups such as N(C₁₋₆alkyl)₂, the alkyl groups may be the same or different.

As used herein, unless otherwise noted, “alkoxy” refers to groups of formula—Oalkyl. Designated numbers of carbon atoms (e.g. −OC₁₋₆) shall refer independently to the number of carbon atoms in the alkoxy group. Non-limiting examples of alkyl groups include methoxy, ethoxy, n-propoxy, iso-propoxy, n-butoxy, sec-butoxy, iso-butoxy, tert-butoxy, and the like. Where so indicated, alkoxy groups can be optionally substituted.

As used herein, the terms “alkenyl” and “alkynyl” groups, whether used alone or as part of a substituent group, refer to straight and branched carbon chains having 2 or more carbon atoms, preferably 2 to 20, having at least one carbon-carbon double bond (“alkenyl”) or at least one carbon-carbon triple bond (“alkynyl”). Where so indicated, alkenyl and alkynyl groups can be optionally substituted. Nonlimiting examples of alkenyl groups include ethenyl, 3-propenyl, 1-propenyl (also 2-methylethenyl), isopropenyl (also 2-methylethe-2-yl), buten-4-yl, and the like. Nonlimiting examples of alkynyl groups include ethynyl, prop-2-ynyl (also propargyl), propyn-1-yl, and 2-methyl-hex-4-yn-1-yl.

As used herein, “cycloalkyl” whether used alone or as part of another group, refers to a non-aromatic hydrocarbon ring including cyclized alkyl, alkenyl, or alkynyl groups, e.g., having from 3 to 14 ring carbon atoms, for example, from 3 to 7 or 3 to 6 ring carbon atoms, and optionally containing one or more (e.g., 1, 2, or 3) double or triple bonds. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Where so indicated, cycloalkyl rings can be optionally substituted. Nonlimiting examples of cycloalkyl groups include: cyclopropyl, cyclopropenyl, cyclobutyl, cyclobutenyl, cyclopentyl, cyclopentenyl, cyclopentadienyl, cyclohexyl, cyclohexenyl, cycloheptyl, cyclooctanyl, decalinyl, octahydropentalenyl, octahydro-1H-indenyl, 3a,4,5,6,7,7a-hexahydro-3H-inden-4-yl, decahydro-azulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen atoms. As used herein, halogen refers to F, Cl, Br and I. Haloalkyl groups include perhaloalkyl groups, wherein all hydrogens of an alkyl group have been replaced with halogens (e.g., —CF₃, —CF₂CF₃). The halogens can be the same (e.g., CHF₂, —CF₃) or different (e.g., CF₂Cl). Where so indicated, haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “aryl” wherein used alone or as part of another group, is defined herein as an aromatic monocyclic ring of 6 carbons or an aromatic polycyclic ring of from 10 to 14 carbons. Aryl groups include but are not limited to, for example, phenyl or naphthyl (e.g., naphthalene-1-yl or naphthalene-2-yl). Where so indicated, aryl groups may be optionally substituted with one or more substituents. Aryl groups also include, but are not limited to for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “heterocycloalkyl” whether used alone or as part of another group, is defined herein as a group having one or more rings (e.g., 1, 2 or 3 rings) and having from 3 to 20 atoms (e.g., 3 to 10 atoms, 3 to 6 atoms) wherein at least one atom in at least one ring is a heteroatom selected from nitrogen (N), oxygen (O), and sulfur (S), and wherein the ring that includes the heteroatom is non-aromatic. In heterocyclyl groups that include 2 or more fused rings, the non-heteroatom bearing ring may be aryl (e.g., indolinyl, tetrahydroquinolinyl, chromanyl). Exemplary heterocycloalkyl groups have from 3 to 14 ring atoms of which from 1 to 5 are heteroatoms independently selected from nitrogen (N), oxygen (O), or sulfur (S). One or more N or S atoms in a heterocycloalkyl group can be oxidized (e.g., N→O⁻, S(O), SO₂). Where so indicated, heterocycloalkyl groups can be optionally substituted.

Non-limiting examples of monocyclic heterocycloalkyl groups include, for example: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic groups having 2 or more rings include, for example: hexahydro-1H-pyrrolizinyl, 3a,4,5,6,7,7a-hexahydro-1H-benzoplimidazolyl, 3a,4,5,6,7,7a-hexahydro-1H-indol yl, 1,2,3,4-tetrahydroquinolinyl, chromanyl, isochromanyl, indolinyl, isoindolinyl, and decahydro-1H-cycloocta[b]pyrrolyl.

The term “heteroaryl” whether used alone or as part of another group, is defined herein as a single or fused ring system having from 5 to 20 atoms (e.g., 5 to 10 atoms, 5 to 6 atoms) wherein at least one atom in at least one ring is a heteroatom selected from nitrogen (N), oxygen (O), and sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzo-thiophenyl, indolyl). Exemplary heteroaryl groups have from 5 to 14 ring atoms and contain from 1 to 5 ring heteroatoms independently selected from nitrogen (N), oxygen (O), and sulfur (S). One or more N or S atoms in a heteroaryl group can be oxidized (e.g., N→O⁻, S(O), SO₂). Where so indicated, heteroaryl groups can be substituted. Non-limiting examples of monocyclic heteroaryl rings include, for example: 1,2,3,4-tetrazolyl, [1,2,3]triazolyl, [1,2,4]triazolyl, triazinyl, thiazolyl, 1H-imidazolyl, oxazolyl, furanyl, thiophenyl, pyrimidinyl, and pyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, and isoquinolinyl.

One non-limiting example of a heteroaryl group as described above is C₁-C₅ heteroaryl, which is a monocyclic aromatic ring having 1 to 5 carbon ring atoms and at least one additional ring atom that is a heteroatom (preferably 1 to 4 additional ring atoms that are heteroatoms) independently selected from nitrogen (N), oxygen (O), and sulfur (S). Examples of C₁-C₅ heteroaryl include, but are not limited to for example, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

For the purposes of the present invention, fused ring groups, spirocyclic rings, bicyclic rings and the like, which comprise a single heteroatom will be considered to belong to the cyclic family corresponding to the heteroatom containing ring. For example, 1,2,3,4-tetrahydroquinoline having the formula:

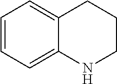

is, for the purposes of the present invention, considered a heterocycloalkyl group. 6,7-Dihydro-5H-cyclopentapyrimidine having the formula:

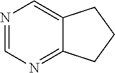

is, for the purposes of the present invention, considered a heteroaryl group. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

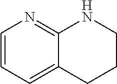

is, for the purposes of the present invention, considered a heteroaryl group.

The term “heteroarylene” whether used alone or as part of another group, is defined herein as a divalent single or fused ring system having from 5 to 20 atoms (e.g., 5 to 10 atoms, 5 to 6 atoms), wherein at least one atom in at least one ring is a heteroatom selected from nitrogen (N), oxygen (O), and sulfur (S), and wherein further at least one of the rings that includes a heteroatom is aromatic. In heteroarylene groups that include 2 or more fused rings, the non-heteroatom bearing ring may be a carbocycle (e.g., 6,7-Dihydro-5H-cyclopentapyrimidinylene) or aryl (e.g., benzofuranylene, benzothiopheylene, indolylene). Exemplary heteroarylene groups have from 5 to 14 ring atoms and contain from 1 to 5 ring heteroatoms independently selected from nitrogen (N), oxygen (O), and sulfur (S). One or more N or S atoms in a heteroarylene group can be oxidized (e.g., N→O⁻, S(O), SO₂). Where so indicated, heteroarylene groups can be substituted. Non-limiting examples of monocyclic heteroarylene rings include, for example: 1,2,3,4-tetrazolylene, [1,2,3]triazolylene, [1,2,4]triazolylene, triazinylene, thiazolylene, 1H-imidazolylene, oxazolylene, furanylene, thiopheneylene, pyrimidinylene, and pyridnylene. Non-limiting examples of heteroarylene rings containing 2 or more fused rings include: benzofuranylene, benzothiopheylene, benzoxazolylene, benzthiazolylene, benztriazolylene, cinnolinylene, naphthyridinylene, phenanthridinylene, 7H-purinylene, 9H-purinylene, 5H-pyrrolo[3,2-d]pyrimidinylene, 7H-pyrrolo[2,3-d]pyrimidinyiene, pyrido[2,3-d]pyrimidinylene, 2-phenylbenzo[d]thiazolylene, 1H-indolylene, 4,5,6,7-tetrahydro-1-H-indolylene, quinoxalinylene, 5-methylquinoxalinylene, quinazolinylene, quinolinylene, and isoquinolinylene.

One non-limiting example of a heteroarylene group as described above is C₁-C₅ heteroarylene, which is a monocyclic aromatic ring having 1 to 5 carbon ring atoms and at least one additional ring atom that is a heteroatom (preferably 1 to 4 additional ring atoms that are heteroatoms) independently selected from nitrogen (N), oxygen (O), and sulfur (S). Examples of C₁-C₅ heteroarylene include, but are not limited to for example, triazinylene, thiazol-2-ylene, thiazol-4-ylene, imidazol-1-ylene; 1H-imidazol-2-ylene, 1H-imidazol-4-ylene, isoxazolin-5-ylene, furan-2-ylene, furan-3-ylene, thiophen-2-ylene, thiophen-4-ylene, pyrimidin-2-ylene, pyrimidin-4-ylene, pyrimidin-5-ylene, pyridin-2-ylene, pyridin-3-ylene, and pyridin-4-ylene.

The term “carbocyclic ring” refers to a saturated cyclic, partially saturated cyclic, or aromatic ring containing from 3 to 14 carbon ring atoms. A carbocyclic ring may be monocyclic, bicyclic or tricyclic. A carbocyclic ring typically contains from 3 to 10 carbon ring atoms and is monocyclic or bicyclic.

The term “heterocyclic ring” refers to a saturated cyclic, partially saturated cyclic, or aromatic ring containing from 3 to 14 ring atoms, in which at least one of the ring atoms is a heteroatom that is oxygen, nitrogen, or sulfur. A heterocyclic ring may be monocyclic, bicyclic or tricyclic. A heterocyclic ring typically contains from 3 to 10 ring atoms and is monocyclic or bicyclic.

The term “amino” refers to —NH₂.

The term “alkylamino” refers to —N(H)alkyl. Examples of alkylamino substituents include methylamino, ethylamino, and propylamino.

The term “dialkylamino” refers to —N(alkyl)₂ where the two alkyls may be the same or different. Examples of dialkylamino substituents include dimethylamino, diethylamino, ethylmethylamino, and dipropylamino.

The term “halogen” refers to fluorine (which may be depicted as —F), chlorine (which may be depicted as —Cl), bromine (which may be depicted as —Br), or iodine (which may be depicted as —I).

The term “azide” refers to —N₃.

The terms “treat” and “treating,” as used herein, refer to partially or completely alleviating, inhibiting, ameliorating and/or relieving a condition from which a patient is suspected to suffer.

As used herein, “therapeutically effective” refers to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, but are not limited to for example, conventional work-ups to determine risk factors that may be associated with the targeted or suspected disease or condition. These and other routine methods allow the clinician to select patients in need of therapy using the methods and compounds of the present invention.

The term “substituted” is used throughout the specification. The term “substituted” is defined herein as a moiety, whether acyclic or cyclic, which has one or more (e.g. 1-10) hydrogen atoms replaced by a substituent as defined herein below. Substituents include those that are capable of replacing one or two hydrogen atoms of a single moiety at a time, and also those that can replace two hydrogen atoms on two adjacent carbons to form said substituent. For example, substituents that replace single hydrogen atoms includes, for example, halogen, hydroxyl, and the like. A two hydrogen atom replacement includes carbonyl, oximino, and the like. Substituents that replace two hydrogen atoms from adjacent carbon atoms include, for example, epoxy, and the like. When a moiety is described as “substituted” any number of its hydrogen atoms can be replaced, as described above. For example, difluoromethyl is a substituted C₁ alkyl; trifluoromethyl is a substituted C₁ alkyl; 4-hydroxyphenyl is a substituted aryl ring; (N,N-dimethyl-5-amino)octanyl is a substituted C₈ alkyl; 3-guanidinopropyl is a substituted C₃ alkyl; and 2-carboxypyridinyl is a substituted heteroaryl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₅, C₃-C₄, C₄-C₆, C₄-C₅, and C₅-C₆ alkyl.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to for example, chiral chromatography, diastereomeric salt formation, kinetic resolution, and asymmetric synthesis. The present invention also includes cis and trans or E/Z isomers of compounds of Formula (I) containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

The term “Liver X receptor (LXR)” as used herein, refers to both LXRα and LXRβ, and variants, isoforms, and active fragments thereof. LXRβ is ubiquitously expressed, while LXRα expression is limited to liver, kidney, intestine, spleen, adipose tissue, macrophages, skeletal muscle, and, as demonstrated herein, skin. Representative GenBank® accession numbers for LXRα sequences include the following: human (Homo sapiens, Q13133), mouse (Mus musculus, Q9Z0Y9), rat (Rattus norvegicus, Q62685), cow (Bos taurus, Q5E9B6), pig (Sus scrofa, AAY43056), chicken (Gallus gallus, AAM90897). Representative GenBank® accession numbers for LXRβ include the following: human (Homo sapiens, P55055), mouse (Mus musculus, Q60644), rat (Rattus norvegicus, Q62755), cow (Bos taurus, Q5BIS6).

The term “mammal” as used herein, refers to a human, a non-human primate, canine, feline, bovine, ovine, porcine, murine, or other veterinary or laboratory mammal. Those skilled in the art recognize that a therapy which reduces the severity of pathology in one species of mammal is predictive of the effect of the therapy on another species of mammal.

The term “modulate” as used herein, refers to encompasses either a decrease or an increase in activity or expression depending on the target molecule. For example, a TIMP1 modulator is considered to modulate the expression of TIMP1 if the presence of such TIMP1 modulator results in an increase or decrease in TIMP1 expression. The term “skin aging” includes conditions derived from intrinsic chronological aging (for example, deepened expression lines, reduction of skin thickness, inelasticity, and/or unblemished smooth surface), those derived from photoaging (for example, deep wrinkles, yellow and leathery surface, hardening of the skin, elastosis, roughness, dyspigmentations (age spots) and/or blotchy skin), and those derived from steroid-induced skin thinning.

Preferred compounds will be LXR modulators with LXRα and/or LXRβ modulator activities. The term “LXR modulator” includes LXRα and/or LXRβ agonists, antagonists and tissue selective LXR modulators, as well as other agents that induce the expression and/or protein levels of LXRs in the skin cells. LXR modulators useful in the present invention include quinoline compounds.

The term “other therapeutic agents” as used herein, refers to any therapeutic agent that has been used, is currently used or is known to be useful for treating a disease or a disorder encompassed by the present invention.

The term “prodrug” as used herein, refers to a pharmacologically inactive derivative of a parent “drug” molecule that requires biotransformation (e.g., either spontaneous or enzymatic) within the target physiological system to release or convert the prodrug into the active drug. Prodrugs are designed to overcome problems associated with stability, toxicity, lack of specificity, or limited bioavailability. Exemplary prodrugs comprise an active drug molecule itself and a chemical masking group (e.g., a group that reversibly suppresses the activity of the drug). Some preferred prodrugs are variations or derivatives of compounds that have groups cleavable under metabolic conditions. Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common prodrugs include acid derivatives such as esters prepared by reaction of parent acids with a suitable alcohol (e.g., a lower alkanol), amides prepared by reaction of the parent acid compound with an amine, or basic groups reacted to form an acylated base derivative (e.g., a lower alkylamide).

The term “pharmaceutically acceptable salt” as used herein, refers to any salt (e.g., obtained by reaction with an acid or a base) of a compound of the present invention that is physiologically tolerated in the target animal (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “therapeutically effective amount” as used herein, refers to that amount of the therapeutic agent sufficient to result in amelioration of one or more symptoms of a disorder, or prevent advancement of a disorder, or cause regression of the disorder. For example, with respect to the treatment of asthma, a therapeutically effective amount preferably refers to the amount of a therapeutic agent that increases peak air flow by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The compounds described herein may be administered to humans and other animals topically in dosage unit formulations containing conventional nontoxic pharmaceutically acceptable carriers, adjuvants, and vehicles as desired. Topical administration may also involve the use of transdermal administration such as transdermal patches or iontophoresis devices.

Methods of formulation are well known in the art and are disclosed, for example, in Remington: The Science and Practice of Pharmacy, Mack Publishing Company, Easton, Pa., 21st Edition (2005), incorporated herein by reference.

Pharmaceutical compositions for use in the present invention can be in the form of sterile, non-pyrogenic liquid solutions or suspensions, coated capsules, suppositories, lyophilized powders, transdermal patches or other forms known in the art.

The choice of carrier(s) and dosage forms will vary with the particular condition for which the composition is to be administered. Examples of various types of preparations for topical/local administration include ointments, lotions, pastes, creams, gels, powders, drops, sprays, solutions, inhalants, patches, suppositories, retention enemas, chewable or suckable tablets or pellets and aerosols. Ointments and creams may, for example, be formulated with an aqueous or oily base with the addition of suitable thickening and/or gelling agents and/or glycols. Such base may thus, for example, include water and/or an oil such as liquid paraffin or a vegetable oil such as arachis oil or castor oil, or a glycolic solvent such as propylene glycol or 1,3-butanediol. Thickening agents which may be used according to the nature of the base include soft paraffin, aluminum stearate, cetostearyl alcohol, polyethylene glycols, woolfat, hydrogenated lanolin and beeswax and/or glyceryl monosterate and/or non-ionic emulsifying agents.

The solubility of the steroid in the ointment or cream may be enhanced by incorporation of an aromatic alcohol such as benzyl alcohol, phenylethyl alcohol or phenoxyethyl alcohol.

Lotions may be formulated with an aqueous or oily base and will in general also include one or more of the following, namely, emulsifying agents, dispersing agents, suspending agents, thickening agents, solvents, coloring agents and perfumes. Powders may be formed with the aid of any suitable powder base e.g. talc, lactose or starch. Drops may be formulated with an aqueous base also comprising one or more dispersing agents, suspending agents or solubilizing agents, etc. Spray compositions may, for example, be formulated as aerosols with the use of a suitable propellane, e.g., dichlorodifluoromethane or tricholorfluoromethane.

The proportion of active ingredient in the compositions according to the invention will vary with the precise compound used, the type of formulation prepared and the particular condition for which the composition is to be administered. The formulation will generally contain from about 0.0001 to about 5.0% by weight of the compound of formula (I). Topical preparations will generally contain 0.0001 to 2.5%, preferably 0.01 to 0.5%, and will be administered once daily, or as needed. Also, generally speaking, the compounds of the invention can be incorporated into topical and other local compositions formulated substantially as are such presently available types of compositions containing known glucocorticosteroids, at approximately the same (or in the case of the most potent compounds of the invention, at proportionately lower) dosage levels as compared to known highly active agents such as methyl prednisolone acetate and beclomethasone dipropionate or at considerably lower dosage levels as compared to less active known agents such as hydrocortisone.

The amount of active ingredient that may be combined with the carrier materials to produce a single dosage form will vary depending upon the host treated and the particular mode of administration. It will be understood, however, that the specific dose level for any particular subject will depend upon a variety of factors including the activity of the specific compound employed, the age, body weight, general health, sex, diet, time of administration, route of administration, rate of excretion, drug combination, and the seventy of the particular disease undergoing therapy. The therapeutically effective amount for a given situation can be readily determined by routine experimentation and is within the skill and judgment of the ordinary clinician.

In another aspect of the invention, kits that include one or more compounds of the invention are provided. Representative kits include a compound described herein (e.g., quinoline esters of Formula I) and a package insert or other labeling including directions for treating skin disorders by administering an effective amount of a compound of the present invention.

In another aspect of the invention, kits that include one or more compounds of the invention are provided. Representative kits include a compound described herein (e.g., quinoline esters of Formula I) and a package insert or other labeling including directions for treating skin disorders in a cell by administering an effective amount of a compound of the present invention.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. A physiologically acceptable carrier should not cause significant irritation to an organism and does not abrogate the biological activity and properties of the administered compound.

An “excipient” refers to an inert substance added to a pharmacological composition to further facilitate administration of a compound. Examples of excipients include but are not limited to calcium carbonate, calcium phosphate, various sugars and types of starch, cellulose derivatives, gelatin, vegetable oils and polyethylene glycols.

A “pharmaceutically effective amount” means an amount which is capable of providing a therapeutic and/or prophylactic effect. The specific dose of compound administered according to this invention to obtain therapeutic and/or prophylactic effect will, of course, be determined by the particular circumstances surrounding the case, including, for example, the specific compound administered, the route of administration, the condition being treated, and the individual being treated. A typical daily dose (administered in single or divided doses) will contain a dosage level of from about 0.01 mg/kg to about 0.50-100 mg/kg of body weight of an active compound of the invention. Preferred daily doses generally will be from about 0.05 mg/kg to about 20 mg/kg and ideally from about 0.1 mg/kg to about 10 mg/kg. Factors such as clearance rate, half-life and maximum tolerated dose (MTD) have yet to be determined but one of ordinary skill in the art can determine these using standard procedures.

As used herein, the term “IC₅₀” refers to an amount, concentration or dosage of a particular test compound that achieves a 50% inhibition of a maximal response in an assay that measures such response. The value depends on the assay used.

As used herein, the term “soft drugs” refer to biologically active chemical compounds (drugs) which might structurally resemble known active drugs (soft analogs) or could be entirely new types of structures, but which are all characterized by a predictable in vivo destruction (metabolism) to nontoxic moieties, after they achieve their therapeutic role. The metabolic disposition of the soft drugs takes place with a controllable rate in a predictable manner.

Soft drug design represents a new approach aimed to design safer drugs with an increased therapeutic index by integrating metabolism considerations into the drug design process. They are designed to be rapidly metabolized into inactive species and, hence, to simplify the transformation-distribution-activity profile of the lead. Consequently, soft drugs are new therapeutic agents obtained by building in the molecule, in addition to the activity, the most desired way in which the molecule is to be deactivated and detoxified subsequent to exerting its biological effects. The desired activity is generally local, and the soft drug is applied or administered near the site of action. Therefore, in most cases, they produce pharmacological activity locally, but their distribution away from the site results in a prompt metabolic deactivation that prevents any kind of undesired pharmacological activity or toxicity.

The major advantages of the soft drug design include:

a) Improvement of the therapeutic index by minimization of undesired systemic side effects and elimination of reactive toxic intermediates;

b) Avoidance of nonlocalized or long-term toxicity by providing an easily accessible route of metabolic degradation;

c) Simplification of the activity/distribution profile by avoiding formation of multiple active species;

d) Elimination of so-called “drug interactions” by avoiding metabolic routes that require competition for saturable, highly used enzyme systems.

The soft drugs of the present invention are quinoline esters of formula (I), which are active upon topical administration and then are hydrolyzed as they pass through the skin into metabolites which, upon absorption into the blood plasma, do not cause serious deleterious effects.

In some embodiments, Z is halogen.

In some embodiments, Z is CF₃.

In some embodiments, Y is alkyl.

In some embodiments, Y is aryl.

In some embodiments, Y is CN.

In some embodiments, Q₁ is H.

In some embodiments, Q₂ is H.

In some embodiments, Q₃ is H.

In some embodiments, Q₃ is halogen.

In some embodiments, L is OC(O).

In some embodiments, L is C(O)O.

In some embodiments, W is H.

In some embodiments, W is halogen.

In some embodiments, W is alkyl.

In some embodiments, X is SO₂Me.

In some embodiments, X is SO₂Et.

In some embodiments, X is SO₂NMe₂.

In some embodiments, X is SO₂NHMe.

In some embodiments, X is alkyl optionally substituted with alkyl, SO₂alkyl or SO₂aryl, or SO₂heteroaryl.

In some embodiments, X is SO₂heteroaryl.

In some embodiments, the compound include:
3-(8-chloro-3-isopropylquinolin-4-yl)phenyl 3-(ethylsulfonyl)benzoate;
3-(8-chloro-3-isopropylquinolin-4-yl)phenyl 2-methyl-5-(methylsulfonyl)benzoate;
3-(8-chloro-3-isopropylquinolin-4-yl)phenyl 4-(methylsulfonyl)benzoate;
3-(8-chloro-3-isopropylquinolin-4-yl)phenyl 2-(methylsulfonyl)benzoate;
3-(8-chloro-3-isopropylquinolin-4-yl)phenyl 2-methyl-5-(methylsulfonyl)benzoate;
3-[3-isopropyl-8-(trifluoromethyl)quinolin-4-yl]phenyl-3-(ethylsulfonyl)benzoate;
3-[3-isopropyl-8-(trifluoromethyl)quinolin-4-yl]phenyl-2-methyl-5-(methylsulfonyl)-benzoate;
3-[3-isopropyl-8-(trifluoromethyl)quinolin-4-yl]phenyl 4-(methylsulfonyl)benzoate;
3-[3-isopropyl-8-(trifluoromethyl)quinolin-4-yl]phenyl 2-(methylsulfonyl)benzoate;
3-[3-isopropyl-8-(trifluoromethyl)quinolin-4-yl]phenyl-5-(dimethylsulfamoyl)-2-methylbenzoate;
3-[3-methyl-8-(trifluoromethyl)quinolin-4-yl]phenyl 3-(methylsulfonyl)benzoate;
3-(methylsulfonyl)phenyl 3-[3-methyl-8-(trifluoromethyl)quinolin-4-yl]benzoate;
3-(8-chloro-3-isopropylquinolin-4-yl)phenyl 2-chloro-5-(methylsulfonyl)benzoate;
3-[3-isopropyl-8-(trifluoromethyl)quinolin-4-yl]phenyl-2-chloro-5-(methylsulfonyl)-benzoate;
3-[3-isopropyl-8-(trifluoromethyl)quinolin-4-yl]phenyl 3-(methylsulfonyl)benzoate;
3-[8-chloro-3-(1-methylethyl)quinolin-4-yl]phenyl 3-(methylsulfonyl)benzoate;
3-(8-chloro-3-methylquinolin-4-yl)phenyl 3-(methylsulfonyl)benzoate;
4-chloro-3-[8-(trifluoromethyl)quinolin-4-yl]phenyl-3-(methylsulfonyl)benzoate;
3-[3-ethyl-8-(trifluoromethyl)quinolin-4-yl]phenyl 3-(methylsulfonyl)benzoate;
3-[3-propyl-8-(trifluoromethyl)quinolin-4-yl]phenyl 3-(methylsulfonyl)benzoate;
3-[8-(trifluoromethyl)quinolin-4-yl]phenyl 3-(methylsulfonyl)benzoate;
3-[3-phenyl-8-(trifluoromethyl)quinolin-4-yl]phenyl 3-(methylsulfonyl)benzoate;
3-[3-benzyl-8-(trifluoromethyl)quinolin-4-yl]phenyl 3-(methylsulfonyl)benzoate;
3-[3-cyano-8-(trifluoromethyl)quinolin-4-yl]phenyl 3-(methylsulfonyl)benzoate;
3-[3-methyl-8-(trifluoromethyl)quinolin-4-yl]phenyl 3-(dimethylsulfamoyl)benzoate;
3-[3-methyl-8-(trifluoromethyl)quinolin-4-yl]phenyl 3-(ethylsulfonyl)benzoate;
3-[3-methyl-8-(trifluoromethyl)quinolin-4-yl]phenyl 2-methyl-5-(methylsulfonyl)benzoate;
3-[3-methyl-8-(trifluoromethyl)quinolin-4-yl]phenyl 2-chloro-5-(methylsulfonyl)benzoate;
3-[3-methyl-8-(trifluoromethyl)quinolin-4-yl]phenyl 4-(methylsulfonyl)benzoate;
3-[3-methyl-8-(trifluoromethyl)quinolin-4-yl]phenyl 5-(dimethylsulfamoyl)-2-methylbenzoate;
3-(8-chloro-3-phenylquinolin-4-yl)phenyl 3-(methylsulfonyl)benzoate;
3-(8-chloro-3-phenylquinolin-4-yl)phenyl 3-(ethylsulfonyl)benzoate;
3-(8-chloro-3-isopropylquinolin-4-yl)phenyl 3-(dimethylsulfamoyl)benzoate;
3-(8-chloro-3-isopropylquinolin-4-yl)phenyl 4-(dimethylsulfamoyl)benzoate;
3-(8-chloro-3-isopropylquinolin-4-yl)phenyl 3-[(methylsulfonyl)methyl]benzoate;
3-(8-chloro-3-isopropylquinolin-4-yl)phenyl 3-(methylsulfamoyl)benzoate;
3-(8-chloro-3-isopropylquinolin-4-yl)phenyl 3-(morpholin-4-ylsulfonyl)benzoate; and
3-(8-chloro-3-isopropylquinolin-4-yl)phenyl 2-methyl-5-(morpholin-4-ylsulfonyl)benzoate; or

a pharmaceutically acceptable salt thereof.

In another embodiments, a pharmaceutical composition comprising a compound of Formula (I) or a pharmaceutically acceptable salt thereof and a pharmaceutically acceptable carrier.

In yet another embodiments, a method of treating a skin disorder in a patient, comprising administering to a patient in need thereof a compound of Formula (I) or a pharmaceutically acceptable salt thereof or a pharmaceutical composition.

In some embodiments, the skin disorder is selected from the group consisting of psoriasis, atopic dermatitis, skin wounds, skin aging, photoaging and wrinkling.

In other embodiments, the treatment of a skin disorder further comprises administering an additional therapeutic agent.

The Liver X receptors (LXR) modulators of the present invention are quinoline esters, and include all enantiomeric and diasteriomeric forms and salts of compounds having the formula (I).

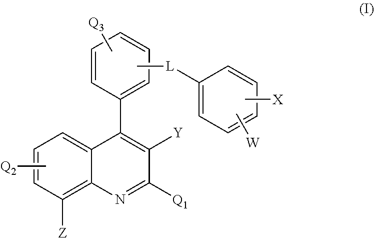

Compounds of the present invention can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given; other process conditions can also be used unless otherwise stated. Optimum reaction conditions can vary with the particular reactants or solvent used. Those skilled in the art will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatography (HPLC), gas chromatography (GC), gel-permeation chromatography (GPC), or thin layer chromatography (TLC).

### Preparation of the Compounds can Involve Protection and Deprotection of Various chemical groups. The chemistry of protecting groups can be found, for example, in Greene et al., Protective Groups in Organic Synthesis, 4th. Ed. (John Wiley & Sons, 2007), the entire disclosure of which is incorporated by reference herein for all purposes.

The reactions or the processes described herein can be carried out in suitable solvents, which can be readily selected by one skilled in the art. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art. The reagents used in the preparation of the compounds of these teachings can be either commercially obtained or can be prepared by standard procedures described in the literature. For example, compounds of the present invention can be prepared according to the methods illustrated in the following Synthetic Schemes.

The description of this invention utilizes a variety of abbreviations well known to those skilled in the art, including the following:

aq.: aqueous

### CH₃CN: Acetonitrile

### DDC: Dicyclohexylcarbodimide

### DCM: Dichloromethane

### DMF: N,N-Dimethylformamide

### DMAP: 4-Dimethylyaminopyridine

### DMSO: Dimethylsulfoxide

EDC: 1-ethyl-3-(3′-dimethylaminopropyl)-carbodimide
EtOAc: Ethyl acetate

### EtOH: Ethanol

### GC: Gas Chromatography

HCl: Hydrochloric acid
HOAc: Acetic acid
HPLC: High performance Liquid Chromatography
K₂CO₃: Potassium carbonate

### MeOH: Methanol

MgSO₄: Magnesium sulfate
NaI: Sodium iodide

### TEA: Triethylamine

TFA: Trifluoroacetic acid

### THF: Tetrahydrofuran

### TLC: Thin Layer Chromatography

### TMS: Trimethylsilyl

### Synthetic Procedures

The reagents used in the preparation of the compounds of this invention can be either commercially obtained or can be prepared by standard procedures described in the literature. In accordance with this invention, compounds in the genus were prepared by following the general schemes.

### General Synthetic Scheme(s) for the Preparation of Intermediates and Compounds of the Invention

According to Scheme 1, the compounds of formula (I) can be prepared be prepared by reacting compounds of formula (1) with benzoic acids of formula (2) under a standard coupling (ester formation) conditions. For example, activation of the acid using dicyclohexylcarbodimide (DCC) or 1-ethyl-3-(3′-dimethylaminopropyl)-carbodimide (EDC), the latter typically in the presence of 4-dimethylaminopyridine (see for example: Dhaon, M. K.; Olsen, R. K.; Ramasamy, K.; Journal of Organic Chemistry, 47, 1962 (1982)).

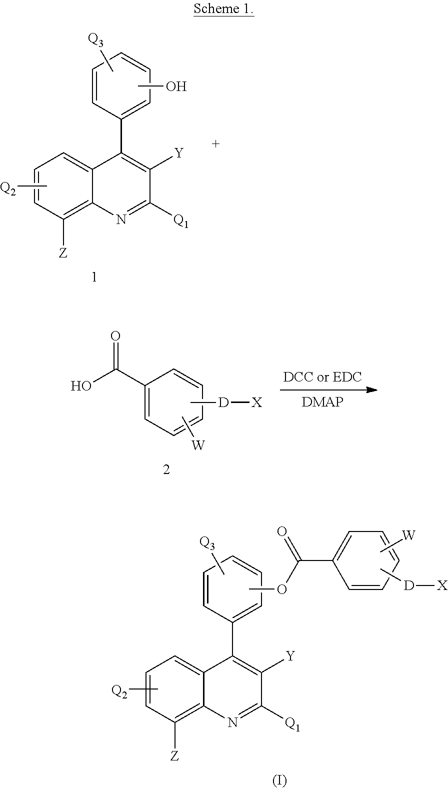

Alternatively, as in Scheme 2, compounds of formula I can be prepared by reaction of an acid chloride of formula 3 with a phenol of formula 1 in the presence of a base, typically triethylamine or diisopropylethylamine, in a solvent such as dichloromethane.

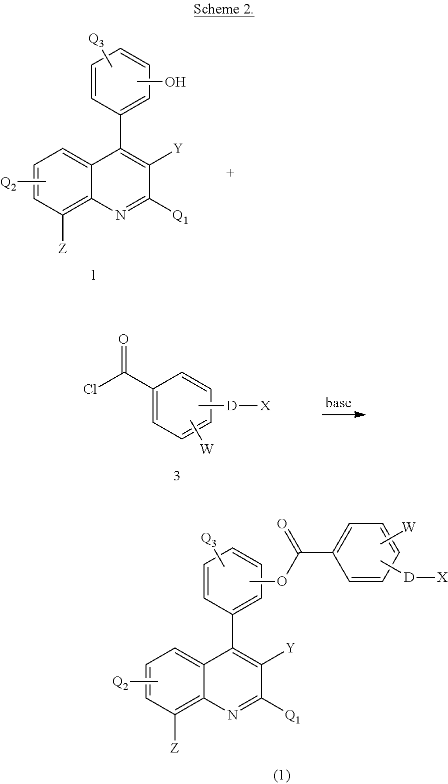

According to Scheme 3, certain compounds of formula (1) in which the carbonyl group of the ester is attached to the 4-phenylquinoline can be prepared by coupling compounds of formula 1 with compounds of formula 4 in essentially the same manner as used in Scheme 1.

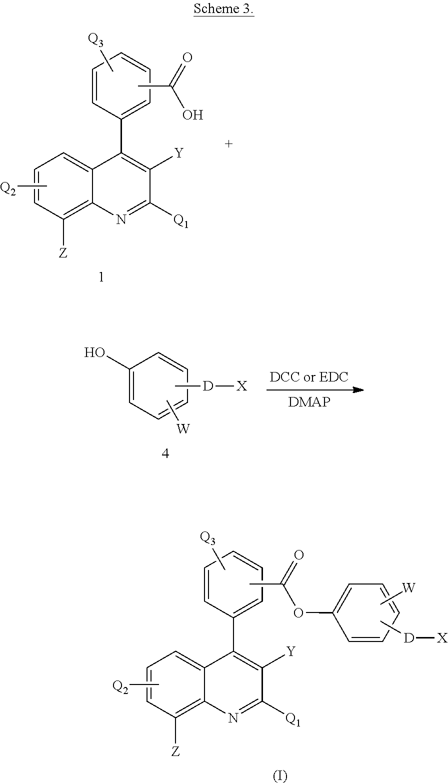

Compounds of formula 1 can be prepared by methods known to one skilled in the art. For example, several preparations of compounds I are described in US . . . and US. One approach involves application of the Friedlander reaction to a mixture of an aminophenone compound of formula 5 and an aldehyde or ketone of formula 6 by heating at an appropriate temperature, typically 80 to 120° C., in an appropriate combination of solvent and strong acid. Examples of such combinations of acid and solvent are benzenesulfonic acid in toluene, sulfuric acid in acetic acid, and the like. If a sensitive or reactive group on compounds of formula 1 was protect during the reaction, for example a phenol may be protected as the methyl ether (methoxy) group, a deprotection step may be performed to remove the protecting group for reaction as in the Schemes above.

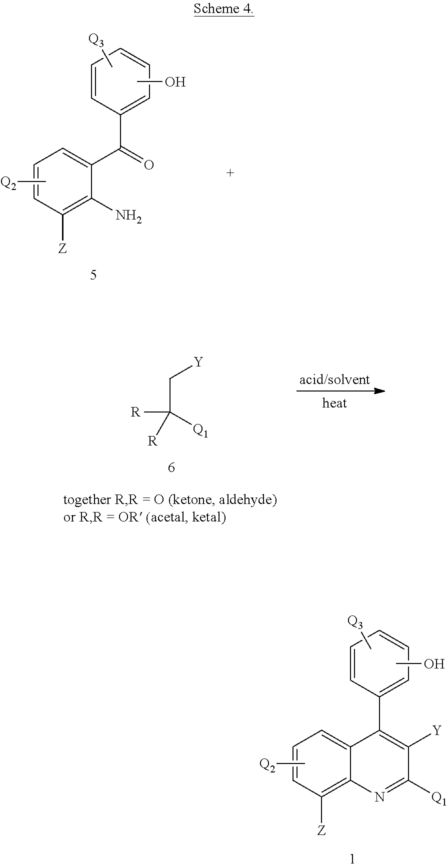

Compounds of formula 2 may be commercially available (e.g., 3-methylsulfonylbenzoic acid) or can be prepared by a variety of methods known to one skilled in the art. For example, as in Scheme 5, reaction of a compound of formula 7 with an amine of formula 8 in a solvent such as dichloromethane, tetrahydrofuran (THE), and the like in the presence of a tertiary organic amine such as triethylamine affords compounds of formula 2 in which D=a bond and X=SO₂NR₂R₃.

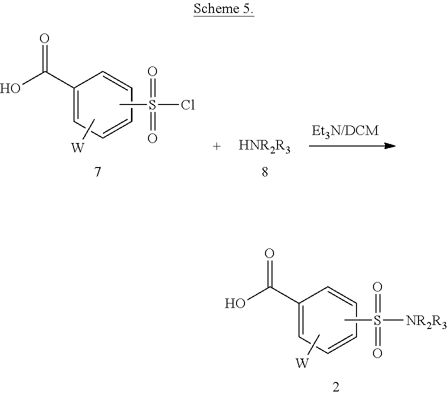

As in Scheme 6, compounds of formula 2 where D=a bond and X=SO₂R₁ can be prepared from compounds of formula 7 by reduction of the sulfonyl chloride to the sulfinic acid salt, typically by heating a mixture of sodium bicarbonate and sodium sulfite in water at 90-100° C. The sulfinic acid salt is alkylated in situ by compounds of formula 9 (R₁-LG) where LG is a leaving group such as a bromide, an iodide, or a sulfonate. Typical alkylating agents include methyliodide, ethyliodide, benzylbromide, and the like. These alkylations are generally performed in the presence of a phase transfer catalyst such as tetrabutylammonium bromide at elevated temperature, up to 100° C., but limited by the boiling point of the alkylating agent.

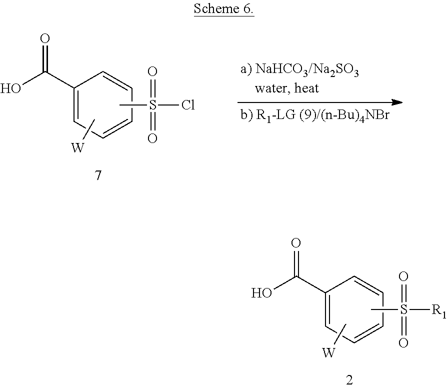

Compounds of formula 7 can be made by sulfonylation of a benzoic acid using chlorosulfonic acid as in Scheme 7 and as described in WO2007/091140 A1, Examples 102 to 105.

### EXAMPLES

The following non-limiting examples are presented merely to illustrate the present invention. The skilled person will understand that there are numerous equivalents and variations not exemplified but which still form part of the present teachings.

The following describes the preparation of representative compounds of this invention. Compounds described as homogeneous were determined to be of 90% or greater purity (exclusive of enantiomers) by analytical reverse phase chromatographic analysis with 254 nM UV detection. Melting points are reported as uncorrected in degrees centigrade. Mass spectral data is reported as the mass-to-charge ratio, m/z; and for high resolution mass spectral data, the calculated and experimentally found masses, [M+H]⁺, for the neutral formulae M are reported.

### Example 1

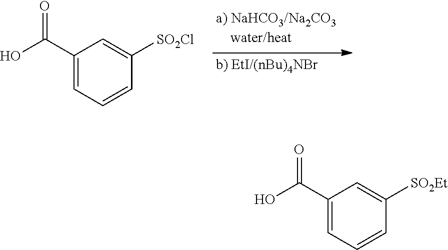

### Step 1: 3-(ethylsulfonyl)benzoic acid A stirred mixture of 3-(chlorosulfonyl)benzoic acid (2.20 g, 10.0 mmol), Na₂SO₃ (2.34 g, 18.5 mmol), and NaHCO₃ (2.52 g, 30.0 mmol) in water (40 mL) was heated at 90° C. for 1 h. The reaction was cooled, treated with ethyliodide (3.45 mL, 50 mmol) and tetrabutylammonium bromide (100 mg), and heated at 80° C. overnight. The reaction was then cooled, extracted with DCM (2×10 mL) to remove excess ethyliodide, and then treated with 2 M aqueous hydrochloric acid until the pH ˜2 was obtained. The resulting solid was suction filtered, washed with water, and vacuum-dried to afford the title compound as an off-white solid (0.99 g). MS (ESI) m/z 213.0.

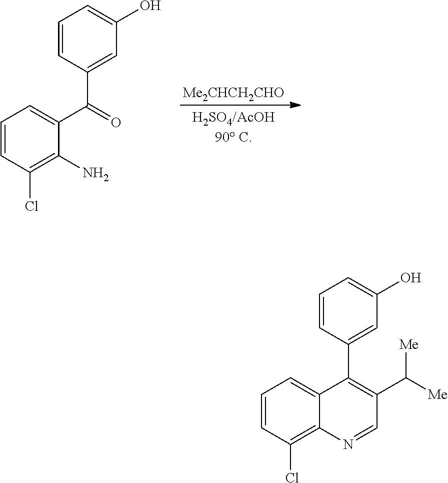

### Step 2: 3-(8-chloro-3-isooropylouinolin-4-yl)phenol

A stirred mixture of (2-amino-3-chlorophenyl)(3-hydroxyphenyl)methanone (2.48 g, 10.0 mmol), hydrocinnamaldehyde (2.58 g, 30.0 mmol), and concentrated sulfuric acid (20 mg) in glacial acetic acid (20 mL) was heated at 90° C. for 48 h. The cooled reaction was poured slowly into a stirred mixture of NaHCO₃ (36g) and water (50 mL). The mixture was extracted with ethyl acetate (2×100 mL) and the dried extracts (MgSO₄) were concentrated in vacuo. The residue was purified by chromatography, eluting with a 0:100 to 35:65 E:H gradient to afford an oil which solidified. Trituration with 10:90 E:H and vacuum drying afforded the title compound as a slightly yellow solid (2.00 g, 67%).

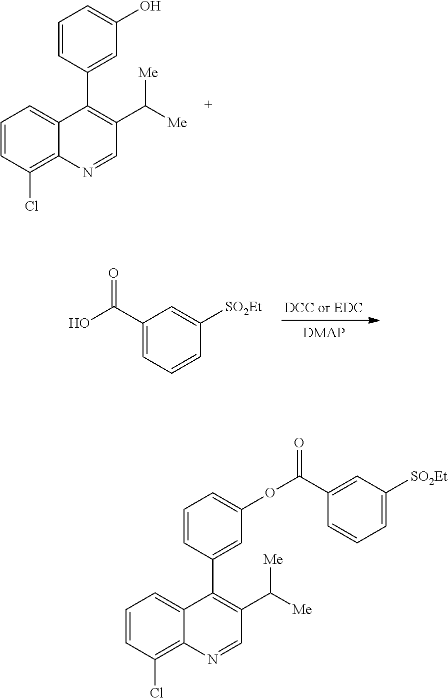

### Step 3: 3-(8-chloro-3-isopromilquinolin-4-yl)phenyl 3-(ethylsulfonyl)benzoate

A stirred mixture of 3-(ethylsulfonyl)benzoic acid (75 mg, 0.35 mmol), 3-(2-propyl)-8-chloro-4-(3-hydroxyphenyl)quinoline (89 mg, 0.30 mmol), and DMAP (20 mg) in DCM (3.0 mL) at 0° C. was treated with 1.0M dicyclohexyldiimide in DCM (0.35 mL, 0.35 mmol). The reaction was allowed to warm to ambient temperature. After 18 h, the reaction was treated with water (5 mL), extracted with DCM, and the combined extracts filtered through a pad of Celite®. The filtrate was dried (MgSO₄), concentrated in vacuo, and then purified by chromatography, eluting with a 30:70 to 70:30 E:H gradient. The product was further purified by reverse phase chromatography eluting with 0:100 to 100:0 CH₃CN:H₂O to remove traces of dicyclohexylurea affording the title compound as a very pale yellow solid (104 mg). MS (ESI) m/z 494.1; HRMS: calcd for C₂₇H₂₄ClNO₄S+H+, 494.1187. Found (ESI, [M+H] Obs'd), 494.1194.

### Example 2

### Step 1: 2-methyl-5-(methylsulfonyl)benzoic acid

The title compound was prepared as in Example 1, step 1, except using 5-(chlorosulfonyl)-2-methylbenzoic acid and methyliodide as the reactants and alkylating at 35° C. Chromatography eluting with 0:100 to 10:90 ethanol:ethyl acetate gave the title compound as a white solid. MS (ESI) m/z 213.0.

### Step 2: 3-(8-chloro-3-isopropyiquinolin-4-yl)phenyl 2-methyl-5-(methylsulfonyl)benzoate

The title compound was prepared as in Example 1, step 3, except using 2-methyl-5-(methylsulfonyl)-benzoic acid to afford the title compound an off-white solid (105 mg). MS (ESI) m/z 494.1; HRMS: calcd for C₂₇H₂₄ClNO₄S+ H+, 494.1187. Found (ESI, [M+H]+Obs'd), 494.1190.

### Example 3

### 3-(8-chloro-3-isopropylquinolin-4-yl)phenyl 4-(methylsulfonyl)benzoate

Prepared as in Example 1, step 3, except using 4-(methylsulfonyl)-benzoic acid to afford the title compound as light yellow solid solid (70 mg). MS (ESI) m/z 480.1; HRMS: calcd for C₂₆H₂₂ClNO₄S+ H+, 480.1031. Found (ESI, [M+H]+ Obs'd), 480.1035.

### Example 4

### 3-(8-chloro-3-isopropylquinolin-4-yl)phenyl 2-(methylsulfonyl)benzoate

Prepared as in Example 1, step 3, except using 2-(methylsulfonyl)-benzoic acid to afford the title compound as light yellow solid (10 mg). MS (ESI) m/z 480.1; HRMS: calcd for C₂₆H₂₂ClNO₄S+ H+, 480.10318. Found (ESI, [M+H]+ Obs'd), 480:1037.

### Example 5

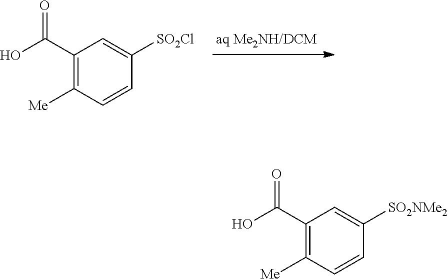

### Step 1: 5-(dimethylsulfamoyl)-2-methylbenzoic acid

A vigorously stirred mixture of 5-(chlorosulfonyl)-2-methylbenzoic acid (1.17 g, 5.00 mmol) in DCM (10 mL) was slowly treated with 40% aqueous diethylamine (2.0 mL). After 18 h at ambient temperature, brine (5 mL) was added and the layers separated. The aqueous was further extracted with DCM (20 mL) and the combined extracts dried (MgSO₄) and concentrated in vacuo. The resulting solid was purified by chromatography eluting with a 50:50 to 100:0 E:gradient to afford the title compound as an off-white solid (0.67 g). MS (ESI) m/z 213.0. MS (ESI) m/z 244.1; HRMS: calcd for C₁₀H₁₃NO₄S+ H+, 244.06380. Found (ESI, [M+H]+ Obs'd), 244.0638.

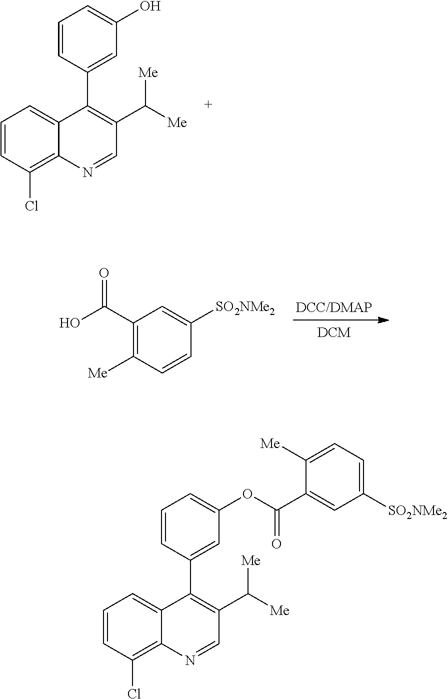

### Step 2: 3-(8-chloro-3-isopropylauinolin-4-yl)phenyl 2-methyl-5-(methylsulfonyl)benzoate

The title compound was prepared as in Example 1, step 3, except using 5-(dimethylsulfamoyl)-2-methylbenzoic acid to afford the title compound as a white solid (128 mg). MS (ESI) m/z 523.2; HRMS: calcd for C₂₈H₂₇ClN₂O₄S+ H+, 523.1453. Found (ESI, [M+H]+ Obs'd), 523.1462.

### Example 6

### 3-[3-isopropyl-8-(trifluoromethyl)quinolin-4-yl]phenyl-3-(ethylsulfonyl)benzoate

The title compound was prepared as in Example 1, step 3, except using 3-[3-isopropyl-8-(trifluoromethyl)quinolin-4-yl]phenol (prepared as in WO 2008049047 A2) as the substrate to give a white solid from a foam (131 mg). MS (ESI) m/z 528.1; HRMS: calcd for C₂₈H₂₄F₃NO₄S+ H+, 528.1451. Found (ESI, [M+H]+Obs'd), 528.1451.

### Example 7

### 3-[3-isopropyl-8-(trifluoromethyl)quinolin-4-yl]phenyl-2-methyl-5-(methylsulfonyl)benzoate

The title compound was prepared as in Example 2, step 2, except using 3-[3-isopropyl-8-(trifluoromethyl)quinolin-4-yl]phenol as the substrate to give a white solid from a foam (109 mg). MS (ESI) m/z 528.1; HRMS: calcd for C₂₈H₂₄F₃NO₄S+ H+, 528.1451. Found (ESI, [M+H] Obs'd), 528.1454.

### Example 8

### 3-[3-isopropyl-8-(trifluoromethyl)quinolin-4-yl]phenyl 4-(methylsulfonyl)benzoate

The title compound was prepared as in Example 3 except using 3-[3-isopropyl-8-(trifluoromethyl)quinolin-4-yl]phenol as the substrate to give a white solid from a foam (98 mg). MS (ESI) m/z 514.1; HRMS: calcd for C₂₇H₂₂F₃NO₄S+ H+, 514.1294. Found (ESI, [M+H]+ Obs'd), 514.1297.

### Example 9

### 3-[3-isopropyl-8-(trifluoromethyl)quinolin-4-yl]phenyl 2-(methylsulfonyl)benzoate

The title compound was prepared as in Example 4 except using 3-[3-isopropyl-8-(trifluoromethyl)quinolin-4-yl]phenol as the substrate to give a white solid from a foam (99 mg). MS (ESI) m/z 514.2; HRMS: calcd for C₂₇H₂₂F₃NO₄S+ H+, 514.1294. Found (ESI, [M+H]+ Obs'd), 514.1308.

### Example 10

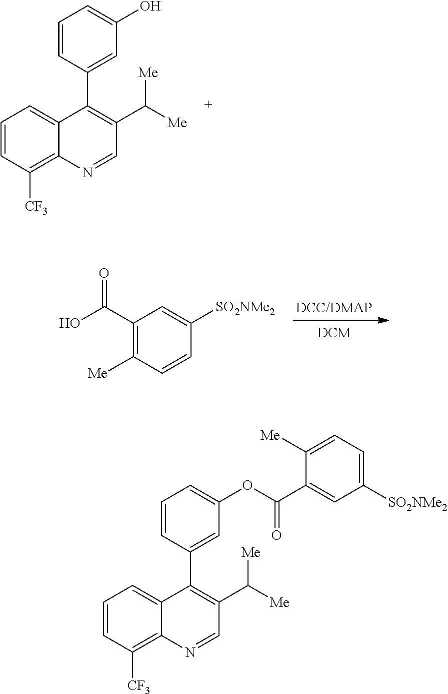

### 3-[3-isopropyl-8-(trifluoromethynauinolin-4-yl]phenyl-5-(dimethylsulfamoyl)-2-methylbenzoate

The title compound was prepared as in Example 5 except using 3-[3-isopropyl-8-(trifluoromethyl)quinolin-4-yl]phenol as the substrate to afford the title compound as a white solid from a foam (139 mg). MS (ESI) m/z 557.2; HRMS: calcd for C₂ₐH₂₇F₃N₂O₄S+ H+, 557.1716. Found (ESI, [M+H]+ Obs'd), 557.1717.

### Example 11

### 3-[3-methyl-8-(trifluoromethynauinolin-4-yl]phenyl 3-(methylsulfonyl)benzoate

A stirred mixture of 3-(methylsulfonyl)benzoic acid (200 mg, 1.00 mmol) in 1,2-dichloroethane (5.0 mL) under nitrogen was treated with thionyl chloride (0.40 mL) and then heated at 80° C. for 2 h. The reaction was cooled slightly and concentrated under a nitrogen stream to remove solvent and excess thionyl chloride to give a white solid. Dichloromethane (10 mL) was added followed by 4-(3-hydroxyphenyl)-3-methyl-8-(trifluoromethyl)quinoline (303 mg, 1.00 mmol). After stirring overnight, the reaction was washed with aqueous saturated NaNCO₃ (5 mL), dried (MgSO₄), and concentrated in vacuo. Chromatography eluting with 0:100 to 40:60 E:H afforded the title compound as a white solid from a foam (372 mg). MS (ESI) m/z 486.1; HRMS: calcd for C₂₅H₁₈F₃NO₄S+H+, 486.0981. Found (ESI, [M+H]+ Obs'd), 486.0982.

### Example 12

### Step 1: 3-[3-methyl-8-(trifluoromethyl)quinolin-4-yl]benzoic acid

A mixture of 4-bromo-3-methyl-8-(trifluoromethyl)quinoline (1.00 g, 3.45 mmol), 3-boronobenzoic acid (0.686 g, 4.14 mmol), tetrakis(triphenylphosphine)palladium (0.199 g, 0.172 mmol) and sodium carbonate (1.096 g, 10.34 mmol) in dioxane (15 ml) and water (5 ml) was refluxed overnight. The reaction was cooled and neutralized with 2N HCl. The mixture was then extracted with ethyl acetate. The combined organics were dried over MgSO₄ and concentrated. Chromatography eluting with 0:100 to 5:95 MeOH:DCM gradient afforded the title compound as a yellow solid (0.818 g, 72%). MS (ESI) m/z 332.1; HRMS: calcd for C₁₈N₁₂F₃NO₂+H+, 332.08929. Found (ESI, [M+H]+ Obs'd), 332.0894

### Step 2: 3-(methylsulfonyl)phenyl 3-[3-methyl-8-(trifluoromethynauinolin-4-yl]benzoate

The title compound was prepared essentially as in Example 11 except using 3-[3-methyl-8-(trifluoromethyl)quinolin-4-yl]benzoic acid as the substrate for conversion to the acid chloride and 3-(methylsulfonyl)phenol as the other reactant, affording a white solid. MS (ESI) m/z 486.1; HRMS: calcd for C₂₅H₁₈F₃NO₄S+ H+, 486.0981. Found (ESI, [M+H] Obs'd), 486.0984.

### Example 13

### 3-(8-chloro-3-isopropylquinolin-4-yl)phenyl 2-chloro-5-(methylsulfonyl)benzoate

A stirred mixture of 2-chloro-5-(methylsulfonyl)benzoic acid (75.5 mg, 0.33 mmol), 3-(2-propyl)-8-chloro-4-(3-hydroxyphenyl)quinoline (89 mg, 0.30 mmol), and DMAP (3.6 mg, 0.03 mmol) in DMF (1.5 mL) at 20° C. was treated with 1-ethyl-3-(3′-dimethylaminopropyl)-carbodiimide hydrochloride (80 mg, 0.45 mmol). After stirring overnight, the reaction was treated with water (10 mL), extracted with ethyl acetate (2×10 mL), and the extracts dried (MgSO₄) and concentrated in vacuo. Purification by chromatography, eluting with a 0:100 to 50:50 E:H gradient gave the title compound as a very pale yellow solid from a foam (67 mg). MS (ESI) m/z 514.1; HRMS: calcd for C₂₆H₂₁Cl₂NO₄S+ H+, 514.0641. Found (ESI, [M+H]+ Obs'd), 514.0640.

### Example 14

### 3-[3-isopropyl-8-(trifluoromethyl)quinolin-4-yl]phenyl-2-chloro-5-(methylsulfonyl)benzoate

The title compound was prepared as in Example 13 except using 3-[3-isopropyl-8-(trifluoromethyl)quinolin-4-yl]phenol as the substrate to give a white solid from a foam (76 mg). MS (ESI) m/z 548.1; HRMS: calcd for C₂₇H₂₁ClF₃NO₄S+ H+, 548.0905. Found (ESI, [M+H] Obs'd), 548.0899.

### Example 15

### 3-[3-isopropyl-8-(trifluoromethyl)quinolin-4-yl]phenyl 3-(methylsulfonyl)benzoate

A stirred mixture of 3-[3-isopropyl-8-(trifluoromethyl)quinolin-4-yl]phenol (99 mg, 0.30 mmol) and 4-methylmorpholine (91 mg, 0.90 mmol) in DCM (3.0 mL) under nitrogen was treated with 3-(methylsulfonyl)benzoic acid chloride (131 mg, 0.60 mmol) and then heated at 35° C. for 21 h. The reaction was cooled, treated with saturated aqueous NaHCO₃ (5 mL) and extracted with DCM (2×3 mL). The combined extracts were dried (MgSO₄), concentrated in vacuo, and the residue purified by chromatography eluting with a 0:100 to 50:50 E:H gradient. The title compound was obtained as a white solid from a foam (84 mg). MS (ESI) m/z 514.1; HRMS: calcd for C₂₇H₂₂F₃NO₄S+ H+, 514.1294. Found (ESI, [M+H]+ Obs'd), 514.1297.

### Example 16

### 3-[8-chloro-3-(1-methylethyl)quinolin-4-yl]phenyl 3-(methylsulfonyl)benzoate

A stirred mixture of 3-(2-propyl)-8-chloro-4-(3-hydroxyphenyl)quinoline (89 mg, 0.30 mmol) and triethylamine (0.20 mL) in DCM (2.0 mL) at 20° C. was treated with 3-(methylsulfonyl)benzoic acid chloride (60 mg, 0.30 mmol). After stirring overnight, the reaction was treated with aqueous NaHCO₃ (3 mL) and extracted with DCM (2×5 mL). The extracts were dried (MgSO₄) and concentrated in vacuo. Purification by chromatography, eluting with a 0:100 to 50:50 E:H gradient gave the title compound as an off-white solid from a foam (50 mg). MS (ESI) m/z 480.1; HRMS: calcd for C₂₆H₂₂ClNO₄S+ H+, 480.1031. Found (ESI, [M+H]+ Obs'd), 480.1036.

### Example 17

### 3-(8-chloro-3-methylquinolin-4-yl)phenyl 3-(methylsulfonyl) benzoate

The title compound was prepared in essentially as in Example 16 except using 8-chloro-4-(3-hydroxyphenyl)-3-methylquinoline as substrate to afford a light yellow solid. MS (ESI) m/z 452.1; HRMS: calcd for C₂₄H₁₈ClNO₄S+ H+, 452.0718. Found (ESI, [M+H]+ Obs'd), 452.0724.

### Example 18

### 4-chloro-3-[8-(trifluoromethyl)quinolin-4-yl]phenyl-3-(methylsulfonyl)benzoate

The title compound was prepared in essentially as in Example 16 except using 4-chloro-3-[(8-(trifluoromethyl)quinolin-4-yl]phenol as substrate to afford a tacky white solid.

MS (ESI) m/z 506.1; HRMS: calcd for C₂₄H₁₅ClF₃NO₄S+ H+, 506.0435. Found (ESI, [M+H]+ Obs'd), 506.0443.

### Example 19

### Step 1: 3-[3-ethyl-8-(trifluoromethyl)quinolin-4-yl]phenol

A mixture of [2-amino-3-(trifluoromethyl)phenyl](3-hydroxyphenyl)methanone (0.200 g, 0.711 mmol), butyraldehyde (0.191 mL, 2.133 mmol), and benzenesulfonic acid (0.337 g, 2.133 mmol) in toluene (3 mL) was refluxed overnight. The reaction was concentrated under a nitrogen stream and taken into ethyl acetate and washed with saturated NaHCO₃, then water. After concentrating in vacuo, the residue was purified by chromatography eluting with a 0:100 to 25:75 E:H gradient to afford the title compound as a brown solid (0.166 g, 74%). MS (ESI) m/z 318.1; HRMS: calcd for C₁₈H₁₄F₃NO+ H+, 318.1100. Found (ESI, [M+H]+ Obs'd), 318.1107.

### Step 2: 3-[3-ethyl-8-(trifluoromethyl)quinolin-4-yl]phenyl 3-(methylsulfonyl)benzoate

The title compound was prepared in essentially as in Example 13 except using 3-[3-ethyl-8-(trifluoromethyl)quinolin-4-yl]phenol as substrate to afford a light yellow solid. MS (ESI) m/z 500.1; HRMS: calcd for C₂₆H₂₀F₃NO₄S++H+, 500.11379. Found (ESI, [M+H]++Obs'd), 500.1139.

### Example 20

### Step 1: 3-[3-propyl-8-(trifluoromethynauinolin-4-yl]phenol

The title compound was prepared as in Example 19, step 1, except using pentanal as the aldehyde substrate to afford a brown solid. MS (ESI) m/z 332.1; HRMS: calcd for C₁₉H₁₆F₃NO+ H+, 332.1257. Found (ESI, [M+H]+ Obs'd), 332.1260.

### Step 2: 3-[3-propyl-8-(trifluoromethyl)quinolin-4-yl]phenyl 3-(methylsulfonyl)benzoate

Prepared as in Example 19, step 2, except using 3-[(8-(trifluoromethyl)quinolin-4-yl]phenol as the substrate to give a white solid. MS (ESI) m/z 514.1; HRMS: calcd for C₂₇H₂₂F₃NO₄S+ H+, 514.12944. Found (ESI, [M+H]+ Obs'd), 514.1292.

### Example 21

### 3-[8-(trifluoromethyl)quinolin-4-yl]phenyl 3-(methylsulfonyl)benzoate

Prepared as in Example 19, step 2, except using 3-[(8-(trifluoromethyl)quinolin-4-yl]phenol as the substrate, to give a yellow solid. MS (ESI) m/z 472.1; HRMS: calcd for C₂₄H₁₆F₃NO₄S+ H+, 472.08249. Found (ESI, [M+H]+ Obs'd), 472.0826.

### Example 22

### 3-[3-phenyl-8-(trifluoromethyl)quinolin-4-yl]phenyl 3-(methylsulfonyl)benzoate

Prepared as in Example 19, step 2, except using 3-[3-phenyl-8-(trifluoromethyl)quinolin-4-yl]phenol as the substrate, to give a light yellow solid. MS (ESI) m/z 548.1; HRMS: calcd for C₃₀H₂₀F₃NO₄S+ H+, 548.1138. Found (ESI, [M+H]+ Obs'd), 548.1139.

### Example 23

### 3-[3-benzyl-8-(trifluoromethyl)quinolin-4-yl]phenyl 3-(methylsulfonyl)benzoate

Prepared as in Example 19, step 2, except using 3-[3-benzyl-8-(trifluoromethyl)quinolin-4-yl]phenol as the substrate, to give a tan solid. MS (ESI) m/z 562.1; HRMS: calcd for C₃₁H₂₂F₃NO₄S+ H+, 562.1294. Found (ESI, [M+H]+ Obs'd), 562.1293.

### Example 24

### 3-[3-cyano-8-(trifluoromethyl)quinolin-4-yl]phenyl 3-(methylsulfonyl)benzoate

Prepared as in Example 19, step 2, except using 3-[3-cyano-8-(trifluoromethyl)quinolin-4-yl]phenol as the substrate, to give an off-white solid. MS (ESI) m/z 497.1; HRMS: calcd for C₂₅H₁₅F₃N₂O₄S+ H+, 497.0777. Found (ESI, [M+H]+ Obs'd), 497.0775.

### Example 25

### 3-[3-methyl-8-(trifluoromethyl)quinolin-4-yl]phenyl 3-(dimethylsulfamoyl)benzoate

Prepared as in Example 13, except using 3-[3-methyl-8-(trifluoromethyl)quinolin-4-yl]phenol and 3-(dimethylsulfamoyl)-benzoic acid as substrates to give a white solid. MS (ESI) m/z 515.1;

### Example 26

### 3-[3-methyl-8-(trifluoromethynauinolin-4-yl]phenyl 3-(ethylsulfonyl)benzoate

Prepared as in Example 13, except using 3-[3-methyl-8-(trifluoromethyl)quinolin-4-yl]phenol and 3-(ethylsulfonyl)benzoic acid as substrates to give a white solid. MS (ESI) m/z 500.1;

### Example 27

### 3-[3-methyl-8-(trifluormethyl)quinolin-4-yl]phenyl 2-methyl-5-(methylsulfonyl)benzoate

Prepared as in Example 13, except using 3-[3-methyl-8-(trifluoromethyl)quinolin-4-yl]phenol and 2-methyl-5-(methylsulfonyl)benzoic acid as substrates to give a white solid. MS (ESI) m/z 500.1;

### Example 28

### 3-[3-methyl-8-(trifluoromethyl)quinolin-4-yl]phenyl 2-chloro-5-(methylsulfonyl)benzoate

Prepared as in Example 13, except using 3-[3-methyl-8-(trifluoromethyl)quinolin-4-yl]phenol and 2-chloro-5-(methylsulfonyl)benzoic acid as substrates to give a white solid. MS (ESI) m/z 520.1;

### Example 29

### 3-[3-methyl-8-(trifluoromethyl)quinolin-4-yl]phenyl 4-(methylsulfonyl)benzoate

Prepared as in Example 13, except using 3-[3-methyl-8-(trifluoromethyl)quinolin-4-yl]phenol and 4-(methylsulfonyl)benzoic acid as substrates to give a white solid. MS (ESI) m/z 486.1;

### Example 30

### 3-[3-methyl-8-(trifluoromethyl)quinolin-4-yl]phenyl 5-(dimethylsulfamoyl)-2-methylbenzoate

Prepared as in Example 13, except using 3-[3-methyl-8-(trifluoromethyl)quinolin-4-yl]phenol and 2-(methylsulfonyl)benzoic acid as substrates to give a white solid from a glass. MS (ESI) m/z 529.1;

### Example 31

### 3-(8-chloro-3-phenylquinolin-4-yl)phenyl 3-(methylsulfonyl)benzoate

Prepared as in Example 13, except using 3-[8-chloro-3-phenylquinolin-4-yl]phenol and 2-(methylsulfonyl)benzoic acid as substrates to give a very pale yellow solid. MS (ESI) m/z 514.1;

### Example 32

### 3-(8-chloro-3-phenylquinolin-4-yl)phenyl 3-(ethylsulfonyl)benzoate

Prepared as in Example 13, except using 3-[8-chloro-3-phenylquinolin-4-yl]phenol and 3-(ethylsulfonyl)benzoic acid as substrates to give a very pale yellow solid. MS (ESI) m/z 528.1;

### Example 33

### Step 1: 3-(dimethylsulfamoyl)benzoic acid

The title compound was prepared essentially as in Example 5, step 1, except using 3-(chlorosulfonyl)benzoic acid chloride and dimethylamine as the substrates to afford an off-white solid. MS (ESI) m/z 252.0; HRMS: calcd for C₉H₁₁NO₄S+ Na+, 252.03010. Found (ESI, [M+N] Obs'd), 252.0297.

### CL-131210-2, L42142-37-1

### Step 2: 3-(8-chloro-3-isopropylauinolin-4-yl)phenyl 3-(dimethylsulfamoyl)benzoate

Prepared as in Example 13, except using 3-(2-propyl)-8-chloro-4-(3-hydroxyphenyl)quinoline and 3-(dimethylsulfamoyl)benzoic acid as the substrates, to give an off-white solid. MS (ESI) m/z 509.1;

### Example 34

### Step 1: 4-(dimethylsulfamoyl)benzoic acid

The title compound was prepared essentially as in Example 5, step 1, except using 4-(chlorosulfonyl)benzoic acid chloride and dimethylamine as the substrates to afford an off-white solid. MS (ESI) m/z 228.0; HRMS: calcd for C₉H₁₁NO₄S+ H+, 230.04815. Found (ESI, [M+H]+ Obs'd), 230.0484.

### CL-131211-2, L42142-37-2

### Step 2: 3-(8-chloro-3-isopropylquinolin-4-yl)phenyl 4-(dimethylsulfamoyl)benzoate

Prepared as in Example 13, except using 3-(2-propyl)-8-chloro-4-(3-hydroxyphenyl)quinoline and 4-(dimethylsulfamoyl)benzoic acid as the substrates, to give an off-white solid. MS (ESI) m/z 509.1;

### Example 35

### Step 1: methyl 3-[(methylsulfonyl)methyl]benzoate

A mixture of methyl 3-(bromomethyl)benzoate (2.29 g, 10.0 mmol) and sodium methylsulfonate (1.25 g, 85% purity, 10.0 mmol based on 85%) in dimethylformamide (10 mL) and water (5 mL) was stirred at 20° C. for 18 h. The reaction was diluted with water (30 mL) and the resulting solid suction filtered, with water washes, and dried under vacuum to afford the title compound as a white solid (2.06 g).

MS (ESI) m/z 246.1; HRMS: calcd for C₁₀H₁₂O₄S+ Na+, 251.03485. Found (ESI, [M+Na]⁺), 251.0350.

### Step 2: 3-[(methylsulfonyl)methyl]benzoic acid

A mixture of methyl 3-[(methylsulfonyl)methyl]benzoate (1.79 g, 8.00 mmol) and 1.0 M aqueous lithium hydroxide (10 mL, 10.0 mmol) in dioxane (10 mL) was stirred at 20° C. for 21 h, then treated with 2.0 M aqueous hydrochloric acid until the pH ca. 2. Additional water was added (10 mL) and the white precipitate was suction filtered, washed with water, and dried under vacuum to afford the title compound as a white solid (1.51 g).

### Step 3: 3-(8-chloro-3-isopropylauinolin-4-yl)phenyl 3-[(methylsulfonyl)methyl]benzoate

Prepared as in Example 13, using 3-(2-propyl)-8-chloro-4-(3-hydroxyphenyl)quinoline and 3-[(methylsulfonyl)methyl]benzoic acid as the substrates, to give an off-white solid.

### Example 36

### Step 1: 3-(methylsulfamoyl)benzoic acid

The title compound was prepared as in Example 5, step 1, except using 3-(chlorosulfonyl)benzoic acid chloride and methylamine (40% aqueous solution) as the substrates to afford an off-white solid.

### Step 2: 3-(8-chloro-3-isopropylquinolin-4-yl)phenyl 3-(methylsulfamoyl)benzoate

Prepared as in Example 13, using 3-(2-propyl)-8-chloro-4-(3-hydroxyphenyl)quinoline and 3-(methylsulfamoyl)benzoic acid as the substrates, to give an off-white solid.

### Example 37

### Step 1: 3-(morpholin-4-ylsulfonyl)benzoic acid

The title compound was prepared as in Example 5, step 1, except using 3-(chlorosulfonyl)benzoic acid and morpholine as the substrates to afford an off-white solid.

MS (ESI) m/z 272.1; HRMS: calcd for C₁₁H₁₃NO₅S+ H+, 272.05872. Found (ESI, [M+H]+ Obs'd), 272.0592.

### Step 2: 3-(8-chloro-3-isopropylauinolin-4-yl)phenyl 3-(morpholin-4-ylsulfonyl)benzoate

Prepared as in Example 13, using 3-(2-propyl)-8-chloro-4-(3-hydroxyphenyl)quinoline and 3-(morpholin-4-ylsulfonyl)benzoic acid as the substrates, to give an off-white solid.

### Example 38

### Step 1: 2-methyl-5-(morpholin-4-ylsulfonyl)benzoic acid

The title compound was prepared as in Example 5, step 1, except using 5-(chlorosulfonyl)-2-methylbenzoic acid and morpholine as the substrates to afford an off-white solid. MS (ESI) m/z 286.1; HRMS: calcd for C₁₂H₁₅NO₅S+ H+, 286.07437. Found (ESI, [M+H]+ Obs'd), 286.0744.

### Step 2: 3-(8-chloro-3-isopropylquinolin-4-yl)phenyl 2-methyl-5-(morpholin-4-ylsulfonyl)benzoate

Prepared as in Example 13, using 3-(2-propyl)-8-chloro-4-(3-hydroxyphenyl)quinoline and 2-methyl-5-(morpholin-4-ylsulfonyl)benzoic acid as the substrates, to give an off-white solid.

### Brief Description of Biological Test Procedure(s) and Text Summary of Results.

### LIGAND-BINDING TEST PROCEDURE FOR HUMAN LXRβ.

Ligand-binding to the human LXRβ was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

### Tracer: ³H T0901317

Receptor source: E. coli extracted from cells expressing biotinylated hLXRβ. Extract was made in a similar buffer as above, but with 50 mM TRIS.

### Day 1

Washed streptavidin and coated flash plates with wash buffer.
Diluted receptor extract to give Bₘₐₓ˜4000 cpm and add to the wells.
Wrapped the plates in aluminum foil and stored them at +4° C. overnight.

### Day 2

Made a dilution series in DMSO of the test ligands.
Made a 5 nM solution of the radioactive tracer in buffer.
Mixed 250 μL diluted tracer with 5 μL of the test ligand from each concentration of the dilution series.
Washed the receptor-coated flash plates.
Added 200 μL per well of the ligand/radiolabel mixture to the receptor-coated flash plates.
Wrapped the plates in aluminum foil and incubate at +4° C. over night.

### Day 3

Aspirated wells, and washed the flashed plates. Sealed the plate.
Measured the remaining radioactivity in the plate.

### Results:

Representative compounds of this invention had activity (IC₅₀ values) in the LXRβ ligand binding assay in the range between 0.001 to 20 uM.

### Summary of Biological Data:

| | | | Gene regulation by LXR | |
|---|---|---|---|---|
| | | | EC₅₀ ABCG1 (nM) | |
| | hLXRb | hLXRa | Human foreskin | |
| Example | binding | binding | fibroblasts | KERTr |
| # | IC₅₀ (uM) | IC₅₀ (uM) | (serum-free) | (serum-free) |
| 1 | 0.0037 | 0.0162 | 59 | 0.76 |
| 2 | 0.0037 | 0.0117 | 33 | 1.46 |
| 3 | 0.017 | 0.057 | 45 | 1.18 |
| 4 | >1 | >1 | | |
| 5 | 0.025 | 0.053 | 50 | 1.62 |
| 6 | 0.0040 | 0.0163 | 37 | 1.48 |
| 7 | 0.0030 | 0.0092 | 23 | 3.34 |
| 8 | 0.0125 | 0.054 | 38 | 1.9 |
| 9 | 0.112 | 0.256 | | |
| 10 | 0.024 | 0.064 | 58 | 0.53 |
| 11 | 0.0035 | 0.0133 | 105 | 73 |
| 12 | 0.154 | 0.544 | | |
| 13 | 0.0034 | 0.0090 | | |
| 14 | 0.0041 | 0.017 | | |
| 15 | 0.0017 | 0.0035 | 41 | 5.2 |
| 16 | 0.0022 | 0.0044 | | |
| 17 | 0.0037 | 0.017 | | |
| 18 | 0.0138 | 0.122 | | |
| 19 | 0.0022 | 0.0040 | 36 | 12.5 |
| 20 | 0.0020 | 0.0029 | 11 | 4.49 |
| 21 | 0.020 | 0.120 | 634 | 384 |
| 22 | 0.0023 | 0.0039 | 9.9 | 9.15 |
| 23 | 0.0027 | 0.0058 | 16 | 25 |
| 24 | 0.033 | 0.090 | | |
| 25 | 0.014 | 0.058 | 250 | 42 |
| 26 | 0.0086 | 0.052 | 250 | 250 |
| 27 | 0.035 | 0.140 | 25 | 99 |
| 28 | 0.019 | 0.105 | 67 | 214 |
| 29 | 0.034 | 0.154 | 250 | 167 |
| 30 | 0.235 | 0.445 | 162 | 137 |
| 31 | 0.0026 | 0.0030 | 0.98 | 2.76 |
| 32 | 0.0030 | 0.0079 | 6.8 | 2.86 |
| 33 | 0.0047 | 0.0150 | 55 | 8.3 |
| 34 | 0.064 | 0.188 | 78 | 1.38 |
| 35 | 0.0056 | 0.0087 | 11 | 0.53 |
| 36 | 0.0042 | 0.0092 | 8.0 | 0.4 |
| 37 | 0.069 | 0.202 | 60 | 2.42 |
| 38 | 0.506 | 0.664 | 44 | 0.62 |

Variations, modifications, and other implementations of what is described herein will occur to those skilled in the art without departing from the spirit and the essential characteristics of the present teachings. Accordingly, the scope of the present teachings is to be defined not by the preceding illustrative description but instead by the following claims, and all changes that come within the meaning and range of equivalency of the claims are intended to be embraced therein.

Each of the printed publications, including but not limited to patents, patent applications, books, technical papers, trade publications and journal articles described or referenced in this specification are herein incorporated by reference in their entirety and for all purposes.

What is claimed is:

1. A compound of Formula (I): 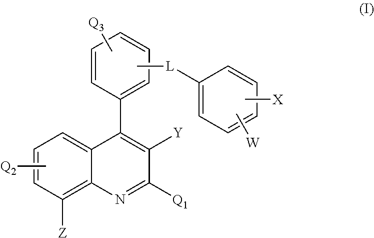
or a pharmaceutically acceptable salt thereof; wherein
Z is halogen or alkyl; wherein each alkyl is optionally substituted with halogen;
Y is H, alkyl, aryl, heteroaryl, cycloalkyl, heterocycloalkyl, CN; wherein each alkyl or aryl is optionally substituted with alkyl, or aryl;
Q₁, Q₂, Q₃ are each independently H, halogen, alkyl, or aryl; wherein each alkyl, or aryl is optionally substituted with alkyl, or aryl;
L is OC(O), C(O)O, CH₂C(O)O, OC(O)CH₂;
W is H, halogen or alkyl;
X is H, alkyl, S(O)ₙR₁, SO₂NR₂R₃, CONR₄R₅, C(R₆)₂OR₇, CN; wherein each alkyl, S(O)ₙR₁, SO₂NR₂R₃, CONR₄R₅, or C(R₆)₂OR₇ is optionally substituted with alkyl,
SO₂alkyl or SO₂aryl, or SO₂heteroaryl; wherein
R₁ is alkyl, aryl, heteroaryl or cycloalkyl;
R₂ and R₃ are each independently H, alkyl or heteroaryl;
R₄ and R₅ are each independently H or alkyl;
R₆ and R₂ are each independently H or alkyl; and
n is 1 or 2.

2. The compound of claim 1, wherein Z is halogen.

3. The compound of claim 1, wherein Z is CF₃.

4. The compound of claim 1, wherein Y is alkyl.

5. The compound of claim 1, wherein Y is aryl.

6. The compound of claim 1, wherein Y is CN.

7. The compound of claim 1, wherein Q₁ is H.

8. The compound of claim 1, wherein Q₂ is H.

9. The compound of claim 1, wherein Q₃ is H.

10. The compound of claim 1, wherein Q₃ is halogen.

11. The compound of claim 1, wherein L is OC(O).

12. The compound of claim 1, wherein L is C(O)O.

13. The compound of claim 1, wherein W is H.

14. The compound of claim 1, wherein W is halogen.

15. The compound of claim 1, wherein W is alkyl.

16. The compound of claim 1, wherein X is SO₂Me.

17. The compound of claim 1, wherein X is SO₂Et.

18. The compound of claim 1, wherein X is SO₂NMe₂.

19. The compound of claim 1, wherein X is SO₂NHMe.

20. The compound of claim 1, wherein X is alkyl optionally substituted with alkyl, SO₂alkyl or SO₂aryl, or SO₂heteroaryl.

21. The compound of claim 1, wherein X is SO₂heteroaryl.

22. The compound of claim 1, wherein the compound is selected from the group consisting of:
3-(8-chloro-3-isopropylquinolin-4-yl)phenyl 3-(ethylsulfonyl)benzoate;
3-(8-chloro-3-isopropylquinolin-4-yl)phenyl 2-methyl-5-(methylsulfonyl)benzoate;
3-(8-chloro-3-isopropylquinolin-4-yl)phenyl 4-(methylsulfonyl)benzoate;
3-(8-chloro-3-isopropylquinolin-4-yl)phenyl 2-(methylsulfonyl)benzoate;
3-(8-chloro-3-isopropylquinolin-4-yl)phenyl 2-methyl-5-(methylsulfonyl)benzoate;
3-[3-isopropyl-8-(trifluoromethyl)quinolin-4-yl]phenyl-3-(ethylsulfonyl)benzoate;
3-[3-isopropyl-8-(trifluoromethyl)quinolin-4-yl]phenyl-2-methyl-5-(methylsulfonyl)-benzoate;
3-[3-isopropyl-8-(trifluoromethyl)quinolin-4-yl]phenyl 4-(methylsulfonyl)benzoate;
3-[3-isopropyl-8-(trifluoromethyl)quinolin-4-yl]phenyl 2-(methylsulfonyl)benzoate;
3-[3-isopropyl-8-(trifluoromethyl)quinolin-4-yl]phenyl-5-(dimethylsulfamoyl)-2-methylbenzoate;
3-[3-methyl-8-(trifluoromethyl)quinolin-4-yl]phenyl 3-(methylsulfonyl)benzoate;
3-(methylsulfonyl)phenyl 3-[3-methyl-8-(trifluoromethyl)quinolin-4-yl]benzoate;
3-(8-chloro-3-isopropylquinolin-4-yl)phenyl 2-chloro-5-(methylsulfonyl)benzoate;
3-[3-isopropyl-8-(trifluoromethyl)quinolin-4-yl]phenyl-2-chloro-5-(methylsulfonyl)-benzoate;
3-[3-isopropyl-8-(trifluoromethyl)quinolin-4-yl]phenyl 3-(methylsulfonyl)benzoate:
3-[8-chloro-3-(1-methylethyl)quinolin-4-yl]phenyl 3-(methylsulfonyl)benzoate;
3-(8-chloro-3-methylquinolin-4-yl)phenyl 3-(methylsulfonyl)benzoate;
4-chloro-3-[8-(trifluoromethyl)quinolin-4-yl]phenyl-3-(methylsulfonyl)benzoate;
3-[3-ethyl-8-(trifluoromethyl)quinolin-4-yl]phenyl 3-(methylsulfonyl)benzoate;
3-[3-propyl-8-(trifluoromethyl)quinolin-4-yl]phenyl 3-(methylsulfonyl)benzoate;
3-[8-(trifluoromethyl)quinolin-4-yl]phenyl 3-(methylsulfonyl)benzoate;
3-[3-phenyl-8-(trifluoromethyl)quinolin-4-yl]phenyl 3-(methylsulfonyl)benzoate;
3-[3-benzyl-8-(trifluoromethyl)quinolin-4-yl]phenyl 3-(methylsulfonyl)benzoate;
3-[3-cyano-8-(trifluoromethyl)quinolin-4-yl]phenyl 3-(methylsulfonyl)benzoate;
3-[3-methyl-8-(trifluoromethyl)quinolin-4-yl]phenyl 3-(dimethylsulfamoyl)benzoate;
3-[3-methyl-8-(trifluoromethyl)quinolin-4-yl]phenyl 3-(ethylsulfonyl)benzoate;
3-[3-methyl-8-(trifluoromethyl)quinolin-4-yl]phenyl 2-methyl-5-(methylsulfonyl)benzoate;
3-[3-methyl-8-(trifluoromethyl)quinolin-4-yl]phenyl 2-chloro-5-(methylsulfonyl)benzoate;
3-[3-methyl-8-(trifluoromethyl)quinolin-4-yl]phenyl 4-(methylsulfonyl)benzoate;
3-[3-methyl-8-(trifluoromethyl)quinolin-4-yl]phenyl 5-(dimethylsulfamoyl)-2-methylbenzoate;
3-(8-chloro-3-phenylquinolin-4-yl)phenyl 3-(methylsulfonyl)benzoate;
3-(8-chloro-3-phenylquinolin-4-yl)phenyl 3-(ethylsulfonyl)benzoate;
3-(8-chloro-3-isopropylquinolin-4-yl)phenyl 3-(dimethylsulfamoyl)benzoate;
3-(8-chloro-3-isopropylquinolin-4-yl)phenyl 4-(dimethylsulfamoyl)benzoate;
3-(8-chloro-3-isopropylquinolin-4-yl)phenyl 3-[(methylsulfonyl)methyl]benzoate;
3-(8-chloro-3-isopropylquinolin-4-yl)phenyl 3-(methylsulfamoyl)benzoate;
3-(8-chloro-3-Isopropylquinolin-4-yl)phenyl 3-(morpholin-4-ylsulfonyl)benzoate; and
3-(8-chloro-3-isopropylquinolin-4-yl)phenyl 2-methyl-5-(morpholin-4-ylsulfonyl)benzoate; or
a pharmaceutically acceptable salt thereof.

23. A pharmaceutical composition comprising a compound of claim 1 or a pharmaceutically acceptable salt thereof and a pharmaceutically acceptable carrier.

24. A method of treating a skin disorder in a patient, comprising administering to a patient in need thereof a compound of claim 1 or a pharmaceutically acceptable salt thereof or a pharmaceutical composition of claim 23.

25. The method of claim 24, wherein the skin disorder is selected from the group consisting of psoriasis, atopic dermatitis, skin wounds, skin aging, photoaging and wrinkling.

26. The method of claim 24, wherein the treatment of a skin disorder further comprises administering an additional therapeutic agent.
